(19) United States
(12) Patent Application Publication
(10) Pub. No.: US 2014/0113833 A1
(43) Pub. Date: Apr. 24, 2014

(54) USE OF MULTIPLE RISK PREDICTORS FOR DIAGNOSIS OF CARDIOVASCULAR DISEASE

(71) Applicant: THE CLEVELAND CLINIC FOUNDATION, Cleveland, OH (US)

(72) Inventor: Stanley L. Hazen, Pepper Pike, OH (US)

(73) Assignee: THE CLEVELAND CLINIC FOUNDATION, Cleveland, OH (US)

(21) Appl. No.: 14/057,106

(22) Filed: Oct. 18, 2013

Related U.S. Application Data
(60) Provisional application No. 61/715,495, filed on Oct. 18, 2012.
(60) Provisional application No. 61/767,483, filed on Feb. 21, 2013.

(51) Int. Cl. G01N 33/68

(52) U.S. Cl. CPC G01N 33/6893
USPC 506/9; 506/12; 506/18

(57) ABSTRACT

Methods and kits for characterizing the risk of developing cardiovascular disease are described. The methods include determining the levels of a plurality of risk predictors selected from the group consisting of B-type natriuretic peptide (BNP), myeloperoxidase (MPO), and high-sensitivity C-reactive protein (hsCRP) predictors in a biological sample from a subject. The levels of the plurality of risk predictors are then compared to corresponding control values to obtain a risk predictor differential for each risk predictor. The plurality of risk predictor differentials are then added to provide a cardiac biomarker score, and the cardiac biomarker score is compared to a reference biomarker score. A positive difference between the cardiac biomarker score and the reference biomarker score indicates the subject has an increased risk of developing cardiovascular disease compared to the risk of a reference population. The methods can be used for risk stratification.

### CROSS-REFERENCE TO RELATED APPLICATIONS

This application claims priority to U.S. Provisional Patent Application No. 61/715,495, filed Oct. 18, 2012, and U.S. Provisional Patent Application No. 61/767,483, filed Feb. 21, 2013, both of which are hereby incorporated by reference in their entirety.

### GOVERNMENT FUNDING

The present invention was made with government support by the National Institutes of Health grants P01HL087018-020001, P01HL076491-055328, 1R01HL103866, 1R01HL103931. The U.S. Government has certain rights in this invention.

### BACKGROUND

Cardiovascular disease (CVD) accounts for one in every two deaths in the United States and is the number one cause of death. Prevention of cardiovascular disease is therefore an area of major public health importance. A low-fat diet and exercise are recommended to prevent CVD. In addition, a number of therapeutic agents may be prescribed by medical professionals to individuals who are known to be at risk for developing or having CVD. More aggressive therapy, such as administration of multiple medications or surgical intervention may be used in those individuals who are at high risk. It is therefore desirable to identify individuals who are at risk, particularly those individuals who are at high risk, of developing or having CVD so that appropriate measures may be taken to reduce the risk for these individuals.

Currently, several risk factors are used by medical professionals to assess an individual's risk of developing or having CVD and to identify individuals at high risk. Major risk factors for cardiovascular disease include age, hypertension, family history of premature CVD, smoking, high total cholesterol, low HDL cholesterol, obesity and diabetes. The major risk factors for CVD are additive, and are typically used together by physicians in a risk prediction algorithm to target those individuals who are most likely to benefit from treatment for CVD. Use of these algorithms in combination with data on risk factors is useful for predicting risk of CVD within 10 years. However, the ability of these methods to identify individuals having a higher probability of developing CVD is limited. Among those individuals with none of the current risk factors, the 10-year risk for developing CVD is still about 2%. In addition, a large number of CVD complications occur in individuals with apparently low to moderate risk profiles, as determined using currently known risk factors. Accordingly, there remains a need for methods to identify a larger spectrum of individuals who are at risk for or affected by CVD.

Increasingly, cardiac biomarkers are also used to provide important information in predicting short-term and long-term risk profiles in patients with acute coronary syndromes. Several clinically available cardiac biomarkers, including B-type natriuretic peptide (BNP), myeloperoxidase (MPO), and high-sensitivity C-reactive protein (hsCRP), provide incremental prognostic value in patients with acute coronary syndromes, alone or in combination. See de Lemos et al., N Engl J Med; 345: 1014-1021 (2001); Brennan et al., N Engl J Med; 349: 1595-1604 (2003); and Ridker et al., N Engl J Med; 342: 836-843 (2000), respectively. Their ability to predict cardiovascular risk has been postulated as they reflect underlying biomarkers of myocardial dysfunction, plaque vulnerability, and systemic inflammation, respectively. Morrow D A, Braunwald E., Circulation; 108: 250-252 (2003). However, the clinical utility of determining these biomarkers simultaneously in a stable non-acute patient cohort has not been established.

### SUMMARY OF THE INVENTION

The inventors hypothesized that simultaneous assessment of these clinically available cardiac biomarkers to produce a risk score (comprised of the sums of “positive biomarkers” based on established cut-off values) would provide incremental prognostic insights into predicting future adverse cardiovascular outcomes. As there is an evolving understanding of diabetes and prediabetes being at heightened cardiovascular risks, the prognostic utility of these cardiac biomarkers across the spectrum of glycemic control was further analyzed.

One aspect of the invention includes a method of characterizing the risk for developing cardiovascular disease. The method includes determining the levels of a plurality of risk predictors in a biological sample obtained from a subject using an analytic device, wherein the risk predictors are selected from the group consisting of B-type natriuretic peptide (BNP), myeloperoxidase (MPO), and high-sensitivity C-reactive protein (hsCRP). The levels of the plurality of risk predictors are then compared to corresponding control values to obtain a risk predictor differential for each risk predictor. The plurality of risk predictor differentials are then added together to provide a cardiac biomarker, and the cardiac biomarker score is compared to a reference biomarker score. A positive difference between the cardiac biomarker score and the reference biomarker score indicates the subject has an increased risk of developing cardiovascular disease compared to the risk of a reference population.

In some embodiments, the amount of the positive difference between the cardiac biomarker score and the reference biomarker score correlates with the level of increased risk of developing cardiovascular disease. In a further embodiment, the method of characterizing the risk for developing cardiovascular disease includes risk stratification, and the risk stratification is obtained by identifying where the subject's cardiac biomarker score falls within a risk profile range. In yet further embodiments, the subject can be diabetic and a risk profile is used, while in other embodiments, the subject is pre-diabetic and a pre-diabetic risk profile is used. In other embodiments, one of the risk profile ranges is a high risk profile, and the method further comprises providing cardiovascular therapeutic invention to a subject identified as having a high risk profile. Cardiovascular therapeutic intervention can include administration of a therapeutic agent, or a beneficial cardiovascular life style change.

Another aspect of the invention provides a kit that includes a plurality of reagents selected from the group consisting of: a reagent capable of detecting B-type natriuretic peptide (BNP), a reagent capable of detecting myeloperoxidase (MPO), and a reagent capable of detecting high-sensitivity C-reactive protein (hsCRP). The kit also includes a plurality of reference values or control samples suitable for use with the selected reagents, and a package holding the reagents. In some embodiments, the kit further includes instructions for using the kit to carry out a method of characterizing the risk for cardiovascular disease for a subject using the reagents and the reference values or control samples. In other embodiments, the reagents are antibodies capable of specifically binding to the compound they are capable of detecting.

### BRIEF DESCRIPTION OF THE FIGURES

The present invention may be more readily understood by reference to the following figures, wherein:

FIG. 1 provides a graph showing a Kaplan-Meier analysis of cardiac biomarker score predicting future major adverse cardiac events at 3-year follow-up.

FIG. 2 provides a graph showing a Forest plot of unadjusted and adjusted Hazard ratios for predicting future major cardiovascular adverse events at 3-year follow-up according to cardiac biomarker score according to subgroups (zero score as reference, adjustments as in Table 3, Model 1).

FIG. 3 provides a graph showing the event rates for future major cardiovascular adverse events at 3-year follow-up according to glycemic status.

### DETAILED DESCRIPTION OF THE INVENTION

The present invention relates to the field of cardiovascular disease. More specifically, it relates to markers and methods for determining whether a subject, particularly a human subject, is at risk of developing cardiovascular disease or experiencing a complication or adverse cardiac event. In particular, the use of a plurality of biomarkers selected from the group including B-type natriuretic peptide (BNP), myeloperoxidase (MPO) and high-sensitivity C-reactive protein (hsCRP) for determining the risk that a subject has or will develop cardiovascular disease is disclosed.

### Definitions

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Prevention or prophylaxis, as used herein, refers to preventing the disease or a symptom of a disease from occurring in a subject which may be predisposed to the disease but has not yet been diagnosed as having it (e.g., including diseases that may be associated with or caused by a primary disease). Prevention may include completely or partially preventing a disease or symptom.

The term therapy, as used herein, encompasses treatment and/or prevention of a disease. The term “intervention” as used herein refers to the specific activity carried out to conduct therapy, and can include use of surgery, life style changes (e.g. change in diet, exercise regime, weight loss, etc.), or the use of one or more therapeutic agents targeted at CVD, (e.g. anti-inflammatory drugs, cholesterol lowering drugs, etc.). Cardiovascular therapeutic intervention refers to therapy directed to treating or preventing cardiovascular disease.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “the BNP” includes reference to one or more BNP molecules and equivalents thereof known to those skilled in the art, and so forth.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

### Diagnostic Methods

The present disclosure provides a method of characterizing the risk of developing cardiovascular disease (CVD), or a complication thereof. The method includes determining the levels of a plurality of risk predictors in a biological sample obtained from a subject using an analytic device, wherein the risk predictors are selected from the group consisting of B-type natriuretic peptide (BNP), myeloperoxidase (MPO), and high-sensitivity C-reactive protein (hsCRP). The levels of the risk predictors are then compared to corresponding control values to obtain a risk predictor differential for each risk predictor. The risk predictor differentials are then added to one another to provide a cardiac biomarker score. The cardiac biomarker score is then compared to a reference biomarker score. A positive difference between the cardiac biomarker score and the reference biomarker score indicates the subject has an increased risk of developing cardiovascular disease compared to the risk of a reference population.

The risk of developing cardiovascular disease refers to the probability that in the future the subject will develop a cardiovascular disease that they currently do not have. Since cardiovascular disease is often the result of the gradual development of a condition, developing cardiovascular disease can also refer to a subject whose cardiovascular condition has worsened to the point where one skilled in the art would recognize it as a disease. The risk that a subject will develop cardiovascular disease can range from 0% to 100% (e.g., 10%, 20%, 30%, etc.). An increased level of risk refers to a percentage increase in the likelihood that cardiovascular disease will develop. Subjects identified as having a high risk of developing cardiovascular disease can be provided with therapeutic intervention to attempt to forestall development of the disease.

The method of characterizing the risk of developing cardiovascular disease, or a complication thereof, uses a plurality of (i.e., two or more) risk predictors to evaluate the risk. The risk predictors used can include B-type natriuretic peptide (BNP), myeloperoxidase (MPO), and high-sensitivity C-reactive protein (hsCRP). For example, the risk predictors can include two risk predictors; e.g., BNP and MPO, BNP and hsCRP, or MPO and hsCRP. Alternately, the method can include use of all three risk predictors; i.e., BNP, MPO, and hsCRP. Determining the levels of BNP, MPO and hsCRP can also include determining the levels of commonly used metabolic precursors or products of these compounds. A metabolic precursor or product, as used herein, refers to a compound that is only one reaction step removed from the primary compound.

B-type natriuretic peptide (BNP), also known as GC-B or brain natriuretic peptide, is a 32 amino acid polypeptide expressed in the heart ventricles and secreted in response to excessive stretch of cardiac myocytes. NT-proBNP is a 76 amino acid N-terminal fragment that is co-secreted with BNP. Plasma concentrations of BNP and NT-pro-BNP are increased in patients with asymptomatic and symptomatic left ventricular dysfunction. NT-proBNP is biologically inactive, but has a biological half-life which is longer than BNP, making it a useful adjunct to analysis of BNP levels. Unless otherwise indicated, reference to determination of the level of BNP is also presumed herein to refer to determination of NT-proBNP, alternately or in addition to the determination of BNP itself.

High sensitivity C-reactive protein (hsCRP) is the most common systemic inflammatory biomarker utilized in clinical practice. High sensitivity CRP is CRP that is detected by highly sensitive methods, but is not an otherwise different molecule. CRP is a 224-residue protein with a monomer molecular mass of 25106 Da. The protein is an annular pentameric disc in shape and a member of the small pentraxins family.

MPO (donor: hydrogen peroxide, oxidoreductase, EC 1.11.1.7) is a tetrameric, heavily glycosylated heme protein of approximately 150 kDa. It is comprised of two identical disulfide-linked protomers, each of which possesses a protoporphyrin-containing 59-64 kDa heavy subunit and a 14 kDa light subunit. See Nauseef, W. M, et al., Blood 67:1504-1507 (1986). Measurement of MPO can include measurement of MPO mass, MPO activity, and/or MPO-generated oxidation products.

The subject's risk of developing cardiovascular disease may occur over a variety of different time frames. For example, the subject may have a risk of developing cardiovascular disease in the long term or the near term. As used herein, the expression “long term” refers to a risk of experiencing a major adverse cardiac event within 10 years. For example, subjects who are at long term risk may be at risk of experiencing a major adverse cardiac event within 1 years, 3 years, 5 years, or 10 years. As used herein, the expression “near term” means within one year. Thus, subjects who are at near term risk may be at risk of experiencing a major adverse cardiac event within the following day, 3 months, or 6 months.

The method of characterizing the risk of developing cardiovascular disease, or a complication thereof, can further include one or more additional steps to obtain further data relating to the risk of developing cardiovascular disease. Examples of additional steps that can be carried out include a) determining the subject's blood pressure; b) determining the levels of low density lipoprotein, cholesterol, apolipoprotein A1, apolipoprotein B100, or creatinine in a biological sample from the subject; c) assessing the subject's response to a stress test; and d) determining the subject's atherosclerotic plaque burden. A reference value may be similarly determined for any other biomarkers, as described herein with respect to determining a reference value for BNP, MPO, and hsCRP. These additional steps can be carried out using procedures known to those skilled in the art. The results of the additional steps are factored into calculation of the cardiac biomarker score.

Also provided herein are methods for monitoring the status of cardiovascular disease in a subject over time. In one embodiment, the method comprises determining the levels of a plurality of risk predictors (i.e., BNP, MPO, and/or hsCRP) in a biological sample taken from the subject at an initial time and in a corresponding biological sample taken from the subject at a subsequent time. For those subjects who have already experienced an acute adverse cardiovascular event such as a myocardial infarction or ischemic stroke, such methods are also useful for assessing the subject's risk of experiencing a subsequent acute adverse cardiovascular event. In such subjects, an increase in levels of the plurality of risk predictors indicates that the subject is at increased risk of experiencing a subsequent adverse cardiovascular event. A decrease in the levels of a plurality of risk predictors in the subject over time indicates that the subject's risk of experiencing a subsequent adverse cardiovascular event has decreased.

### Methods for Measuring Levels of Risk Predictors

The levels of risk predictors can be measured by an analytic device such as a kit or a conventional laboratory apparatus, which can be either a portable or a stationary device. The analytic device may be a spectrometric device, such as a mass spectrometer, an ultraviolet spectrometer, or a nuclear magnetic resonance spectrometer, or an immunoassay. A spectrometer is a device that uses a spectroscopic technique to assess the concentration or amount of a given species in a medium such as a biological sample (e.g., a bodily fluid). In addition to including equipment used for detecting the level of the risk predictor, the analytic device can also include additional equipment to provide physical separation of analytes prior to analysis (i.e., a separation device). For example, if the analyte detector is a mass spectrometer, it may also include a high performance liquid chromatograph (HPLC) or gas chromatograph (GC) to purify the risk predictor before its detection by mass spectrometry. The separation device and the analyte detector may be provided and referred to as a single device; e.g., HPLC with on-line electrospray ionization tandem mass spectrometry. Other methods to detect biomarkers include, e.g., fluorometry, colorimetry, radiometry, luminometry, or other spectrometric methods, plasmon-resonance, and one- or two-dimensional gel electrophoresis. In some embodiments, the levels of the risk predictors may be compared to the level of corresponding internal standards in the sample or samples when carrying out the analysis to characterize and/or quantify the compounds being detected.

While there is considerable overlap with regard to the analytic devices that can be used to determine the level of risk predictors, certain analytic devices may be preferred depending on the specific risk predictor being evaluated. For example, mass spectrometry-based methods are preferred for determining the amounts of smaller metabolites such as lipid oxidation products resulting from MPO activity, whereas immunoassays are generally preferred for larger risk predictors such as proteins. For a detailed discussion of suitable analytic methods for evaluating MPO mass, MPO activity and MPO-generated oxidation products, see U.S. Pat. No. 7,459,286, the disclosure of which is incorporated herein by reference.

Various analytical methods are available for CRP determination, such as ELISA, immunoturbidimetry, rapid immunodiffusion, and visual agglutination. A high-sensitivity CRP (hs-CRP) test measures low levels of CRP using laser nephelometry. Laser nephelometry is performed by measuring the turbidity in a water sample by passing laser light through the sample being measured. In nephelometry the measurement is made by measuring the light passed through a sample at an angle.

Various methods are also known to those skilled in the art for determining BNP and/or NT-proBNP levels, with immunoassays being a preferred method. Commercially available assays cleared for measurement of BNP are sandwich-type immunoassay methods based on two monoclonal antibodies or a combination of monoclonal and polyclonal antibodies. Sandwich-type NT-proBNP immunoassays have also been cleared by the FDA and worldwide for routine application. A comparative review of BNP and NT-proBNP immunoassays is provided by Clerico et al. (Clerico et al., Clin Chem., 53(5): 813-22 (2007)).

As indicated herein, mass spectrometry-based methods can be used to assess level of one or more risk predictors in a biological sample. Mass spectrometers include an ionizing source (e.g., electrospray ionization), an analyzer to separate the ions formed in the ionization source according to their mass-to-charge (m/z) ratios, and a detector for the charged ions. In tandem mass spectrometry, two or more analyzers are included. Such methods are standard in the art and include, for example, HPLC with on-line electrospray ionization (ESI) and tandem mass spectrometry.

Other spectrometric methods can also be used to detect risk predictors. For example, risk predictors can be measured by HPLC using a variety of detectors including, but not limited to UV or Vis (of a derivatized form), mass spectrometry, or GC/MS. Another method that can be used to identify risk predictors is nuclear magnetic resonance (NMR). Examples of NMR include proton NMR and carbon-13 NMR.

Levels of risk predictors in a biological sample can be determined using polyclonal or monoclonal antibodies that are immunoreactive with the risk predictors. For example, antibodies immunospecific for myeloperoxidase may be made and labeled using standard procedures and then employed in immunoassays to detect the presence of myeloperoxidase in a sample. Suitable immunoassays include, by way of example, immunoprecipitation, particle immunoassay, immunonephelometry, radioimmunoassay (RIA), enzyme immunoassay (EIA) including enzyme-linked immunosorbent assay (ELISA), sandwich, direct, indirect, or competitive ELISA assays, enzyme-linked immunospot assays (ELISPOT), fluorescent immunoassay (FIA), chemiluminescent immunoassay, flow cytometry assays, immunohistochemistry, Western blot, and protein-chip assays using for example antibodies, antibody fragments, receptors, ligands, or other agents binding the target analyte. Polyclonal or monoclonal antibodies raised against suitable risk predictors are produced according to established procedures. Generally, for the preparation of polyclonal antibodies, a protein or peptide fragment thereof is used as an initial step to immunize a host animal. A general review of immunoassays is available in Methods in Cell Biology v. 37: Antibodies in Cell Biology, Asai, ed. Academic Press, Inc. New York (1993), and Basic and Clinical Immunology 7th Ed., Stites & Ten, eds. (1991).

Antibodies may be used to detect the presence, or measure the amount of a risk predictor in a biological sample from the subject. Use of antibodies comprises contacting a sample taken from the individual with one or more of the antibodies; and assaying for the formation of a complex between the antibody and a protein or peptide in the sample. For ease of detection, the antibody can be attached to a substrate such as a column, plastic dish, matrix, or membrane, preferably nitrocellulose. The sample may be untreated, subjected to precipitation, fractionation, separation, or purification before combining with the antibody. Interactions between antibodies in the sample and the risk predictor are detected by radiometric, colorimetric, or fluorometric means, size-separation, or precipitation. Preferably, detection of the antibody-protein or peptide complex is by addition of a secondary antibody that is coupled to a detectable tag, such as for example, an enzyme, fluorophore, or chromophore. Formation of the complex is indicative of the presence of the risk predictor in the subject's biological sample.

Once the levels of one or more risk predictors (i.e., BNP, MPO, and hsCRP) have been determined, they can be displayed in a variety of ways. For example, the levels of the risk predictors can be displayed graphically on a display as numeric values or proportional bars (i.e., a bar graph) or any other display method known to those skilled in the art. The graphic display can provide a visual representation of the amount of the risk predictor in the biological sample being evaluated. In addition, in some embodiments, the analytic device can also be configured to display the risk predictor or a comparison of the level of risk predictor to a control value based on levels of the risk predictor in comparable bodily fluids from a reference cohort.

In another embodiment, a system (e.g., computer system and/or software) that is configured to receive patient data related to BNP, MPO, and/or hsCRP levels, and optionally other patient data (e.g., related to other CVD risk factors and markers) and to calculate and display a risk score is provided. In some such embodiments, the system employs one or more algorithms to convert the biological data into a risk score. In some embodiments, the system comprises a database that associates marker levels with risk profiles, based, for example, on historic patient data, one or more control subjects, population averages, or the like. In some embodiments, the system comprises a user interface that permits a user to manage the nature of the information assessed and the manner in which the risk score is displayed. In some embodiments, the system comprises a display that displays a risk score to the user.

### Biological Samples

“Biological sample” as used herein is meant to include any biological sample from a subject where the sample is suitable for analysis of one or more of the risk factors. Suitable biological samples for determining the levels of BNP, MPO, and/or hsCRP in a subject include but are not limited to bodily fluids such as blood-related samples (e.g., whole blood, serum, plasma, and other blood-derived samples), urine, sputem, cerebral spinal fluid, bronchoalveolar lavage, and the like. Another example of a biological sample is a tissue sample. Risk factor levels can be assessed either quantitatively or qualitatively, usually quantitatively. The levels of the risk factors can be determined either in vitro or ex vivo.

The methods involve providing or obtaining a biological sample from the subject, which can be obtained by any known means including needle stick, needle biopsy, swab, and the like. In an exemplary method, the biological sample is a blood sample, which may be obtained for example by venipuncture.

A biological sample may be fresh or stored. Biological samples may be or have been stored or banked under suitable tissue storage conditions. The biological sample may be a bodily fluid expressly obtained for the assays of this invention or a bodily fluid obtained for another purpose which can be subsampled for the assays of this invention. Preferably, biological samples are either chilled or frozen shortly after collection if they are being stored to prevent deterioration of the sample.

In one embodiment, the biological sample is whole blood. Whole blood may be obtained from the subject using standard clinical procedures. In another embodiment, the biological sample is plasma. Plasma may be obtained from whole blood samples by centrifugation of anti-coagulated blood. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions at physiological pH, such as phosphate, Tris, or the like, can be used.

### Subjects

The terms “individual,” “subject,” and “patient” are used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Treatment of humans is of particular interest.

The subject is any human or other animal to be tested for characterizing its risk of CVD. In certain embodiments, the subject does not otherwise have an elevated risk of an adverse cardiovascular event. Subjects having an elevated risk of an adverse cardiovascular event include those with a family history of cardiovascular disease, elevated lipids, smokers, prior acute cardiovascular event, etc. (See, e.g., Harrison's Principles of Experimental Medicine, 15th Edition, McGraw-Hill, Inc., N.Y.—hereinafter “Harrison's”).

In certain embodiments the subject is apparently healthy. “Apparently healthy”, as used herein, describes a subject who does not have any signs or symptoms of CVD or has not previously been diagnosed as having any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. Other biomarkers can be used to identify subjects who are apparently healthy. For example, in some embodiments it is useful to conduct a diagnosis of subjects who are troponin-negative, which would generally indicate that the subject is not currently at high risk of a heart attack.

In certain embodiments, the subject is a nonsmoker. “Nonsmoker” describes an individual who, at the time of the evaluation, is not a smoker. This includes individuals who have never smoked as well as individuals who have smoked but have not smoked tobacco products within the past year. In certain embodiments, the subject is a smoker.

Glycemic status is another important risk factor for cardiovascular disease. Accordingly, in additional embodiments, the subject is diabetic or pre-diabetic. Glycemic status and clinical definitions of diabetes mellitus, “pre-diabetes,” and non-diabetes are defined by the latest practice guidelines based on fasting glucose and glycated hemoglobin levels (fasting glucose<100 mg/dL and HbA1c<5.7% for normals; fasting glucose≧126 mg/dL or HbA1c≧6.5% or currently taking glucose-lowering medications for diabetes mellitus; neither normal nor diabetes mellitus for pre-diabetes). See Standards of medical care in diabetes—Diabetes Care, 35 Suppl 1:211-63 (2012).

### Cardiovascular Disease

The present disclosure provides a method of characterizing the risk of developing cardiovascular disease. As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to myocardial infarction, acute coronary syndrome, angina, congestive heart failure, aortic aneurysm, aortic dissection, iliac or femoral aneurysm, pulmonary embolism, atrial fibrillation, stroke, transient ischemic attack, systolic dysfunction, diastolic dysfunction, myocarditis, atrial tachycardia, ventricular fibrillation, endocarditis, peripheral vascular disease, and coronary artery disease (CAD).

A cardiovascular event, as used herein, refers to the manifestation of an adverse condition in a subject brought on by cardiovascular disease, such as sudden cardiac death or acute coronary syndromes including, but not limited to, myocardial infarction, unstable angina, aneurysm, or stroke. The term “cardiovascular event” can be used interchangeably herein with the term cardiovascular complication. Because diseases are often referred to by the complications that result therefrom, there is significant overlap in the terms used for cardiovascular disease and cardiovascular complications. While a cardiovascular event can be an acute condition (i.e., a brief and typically severe condition), it can also represent the worsening of a previously detected condition to a point where it represents a significant threat to the health of the subject, such as the enlargement of a previously known aneurysm or the increase of hypertension to life threatening levels. Examples of cardiovascular complications include heart failure, non-fatal myocardial infarction, stroke, angina pectoris, transient ischemic attacks, aortic aneurysm, aortic dissection, cardiomyopathy, abnormal cardiac catheterization, abnormal cardiac imaging, stent or graft revascularization, risk of experiencing an abnormal stress test, risk of experiencing abnormal myocardial perfusion, and death.

The presence of cardiovascular disease can be confirmed using a variety of techniques known to those skilled in the art. Medical procedures for determining whether a human subject has coronary artery disease or is at risk for experiencing a complication of coronary artery disease include, but are not limited to, coronary angiography, coronary intravascular ultrasound (IVUS), stress testing (with and without imaging), assessment of carotid intimal medial thickening, carotid ultrasound studies with or without implementation of techniques of virtual histology, coronary artery electron beam computer tomography (EBTC), cardiac computerized tomography (CT) scan, CT angiography, cardiac magnetic resonance imaging (MRI), and magnetic resonance angiography (MRA).

In some embodiments, the cardiovascular disease is an acute coronary syndrome. Acute coronary syndrome (ACS) refers to cardiovascular disease attributed to obstruction of the coronary arteries. The most common symptom prompting diagnosis of ACS is chest pain, often radiating of the left arm or angle of the jaw, pressure-like in character, and associated with nausea and sweating. Acute coronary syndrome usually occurs as a result of one of three problems: ST elevation myocardial infarction (30%), non ST elevation myocardial infarction (25%), or unstable angina (38%). ACS is distinguished from stable angina, which develops during exertion and resolves at rest. In contrast with stable angina, unstable angina occurs suddenly, often at rest or with minimal exertion, or at lesser degrees of exertion than the individual's previous angina (“crescendo angina”). New onset angina is also considered unstable angina, since it suggests a new problem in a coronary artery.

As used herein, the phrase “major adverse cardiovascular event” (MACE) is defined as the occurrence of heart failure, aortic dissection, aortic aneurism, non-fatal myocardial infarction, non-fatal stoke, or death for a subject within 3 years of evaluation of the subject.

Heart failure is a form of cardiovascular disease is a condition in which a problem with the structure or function of the heart impairs its ability to supply sufficient blood flow to meet the body's needs, characterized by compromised ventricular systolic or diastolic functions, or both. Heart failure may be manifested by symptoms of poor tissue perfusion alone (e.g., fatigue, poor exercise tolerance, or confusion) or by both symptoms of poor tissue perfusion and congestion of vascular beds (e.g., dyspnea, chest rates, pleural effusion, pulmonary edema, distended neck veins, congested liver, or peripheral edema). Congestive heart failure represents a form of heart failure where cardiac output is low, in contrast with high output cardiac failure, in which the body's requirements for oxygen and nutrients are increased, and demand outstrips what the heart can provide.

Heart failure can occur as a result of one or more causes. A major cause is secondary atherosclerotic disease, where one or more ischemic events such as a heart attack result in ischemic injury to the heart and decreased function. This type of heart failure is referred to as ischemic heart failure, because the cause of the cardiac dysfunction was secondary to the ischemic injury. Ischemic heart failure can also result from other cardiovascular conditions leading to ischemic injury, such as atherosclerosis that limits blood flow.

Heart failure can also occur as a result of causes other than ischemia, and such forms of heart failure are referred to as non-ischemic heart failure. Examples of non-ischemic heart failure include myocarditis resulting from viral infection, amyloidosis of cardiac tissue, arrhythmia, manifestation of genetic defects, injury from abuse of alcohol, drugs, or cigarettes, other sources of injury to cardiac tissue such as infection by bacteria or parasites, or vitamin deficiency.

Aortic dissection is a tear in the wall of the aorta that causes blood to flow between the layers of the wall of the aorta and force the layers apart. In an aortic dissection, blood penetrates the intima, which is the innermost layer of the aortic artery, and enters the media layer. The high pressure rips the tissue of the media apart along the laminated plane splitting the inner ⅔ and the outer ⅓ of the media apart. This can propagate along the length of the aorta for a variable distance forward or backwards. Dissections that propagate towards the iliac bifurcation (with the flow of blood) are called anterograde dissections and those that propagate towards the aortic root (opposite of the flow of blood) are called retrograde dissections. The initial tear is usually within 100 mm of the aortic valve so a retrograde dissection can easily compromise the pericardium leading to a hemocardium. Aortic dissection is a severe cardiovascular complication and can quickly lead to death, even with optimal treatment.

Symptoms of aortic dissection are known to those skilled in the art, and include severe pain that had a sudden onset that may be described as tearing in nature, or stabbing or sharp in character. Some individuals will report that the pain migrates as the dissection extends down the aorta. While the pain may be confused with the pain of a myocardial infarction, aortic dissection is usually not associated with the other signs that suggest myocardial infarction, including heart failure, and ECG changes. Individuals experiencing an aortic dissection usually do not present with diaphoresis (profuse sweating). Individuals with chronic dissection may not indicate the presence of pain. Aortic insufficiency is also typically seen. Other less common symptoms that may be seen in the setting of aortic dissection include congestive heart failure (7%), syncope (9%), cerebrovascular accident (3-6%), ischemic peripheral neuropathy, paraplegia, cardiac arrest, and sudden death. Preferably, this diagnosis is made by visualization of the intimal flap on a diagnostic imaging test such as a CT scan of the chest with iodinated contrast material and a trans-esophageal echocardiogram.

An aortic aneurysm, on the other hand, is a cardiovascular disorder characterized by a swelling of the aorta, which is usually caused by an underlying weakness in the wall of the aorta at that location. Aortic aneurysms are classified by where they occur on the aorta. Abdominal aortic aneurysms, hereafter referred to as AAAs, are the most common type of aortic aneurysm, and are generally asymptomatic before rupture. The most common sign for the aortic aneurysm is the Erythema Nodosum also known as leg lesions typically found near the ankle area. AAAs are attributed primarily to atherosclerosis, though other factors are involved in their formation. An AAA may remain asymptomatic indefinitely. There is a large risk of rupture once the size has reached 5 cm, though some AAAs may swell to over 15 cm in diameter before rupturing. Only 10-25% of patients survive rupture due to large pre- and post-operative mortality.

Symptoms of an aortic aneurysm may include: anxiety or feeling of stress; nausea and vomiting; clammy skin; rapid heart rate. However, an intact aortic aneurysm may not produce symptoms. As they enlarge, symptoms such as abdominal pain and back pain can develop. Compression of nerve roots may cause leg pain or numbness. Untreated, aneurysms tend to become progressively larger, although the rate of enlargement is unpredictable for a given individual. In some cases, clotted blood which lines most aortic aneurysms can break off and result in an embolus. Preferably, medical imaging is used to confirm the diagnosis of an aortic aneurysm.

### Comparison of Risk Predictor Levels to Corresponding Control Values

A method of characterizing the risk for developing cardiovascular disease is disclosed. The method includes comparing the levels of a plurality of risk predictors in a biological sample obtained from a subject to corresponding control values to obtain a risk predictor differential for each risk predictor. The risk predictors are selected from the group consisting of B-type natriuretic peptide (BNP), myeloperoxidase (MPO), and high-sensitivity C-reactive protein (hsCRP). The corresponding control values are therefore a BNP control value, an MPO control value, and an hsCRP control value. The risk predictor differential represents the difference between the level of the risk predictor found in the biological sample and the corresponding control value, and is determined by subtracting the control value from the level of the risk predictor in the biological sample. For example, the risk predictor differential for B-type natriuretic peptide is determined by subtracting the BNP control value from the BNP level found in the biological sample.

A risk predictor differential is calculated for each of the risk predictors being evaluated. Thus, depending on the risk predictors being evaluated, there can be a BNP risk predictor differential, a MPO risk predictor differential, and an hsCRP risk predictor differential. The plurality of risk predictor differentials are then added to provide an overall a cardiac biomarker score. In some embodiments, the risk predictor differentials are simplified to binary numbers. In this embodiment, each risk predictor that exceeds the control value is designated a positive risk predictor having a risk predictor differential of 1 and all other risk predictors are designated as null risk predictors having a risk predictor differential of 0. In this embodiment, the cardiac biomarker score will be an integer from 0 to 3, depending on the number of risk predictors used and how many of them were determined to be positive risk predictors.

Finally, the cardiac biomarker score is compared to a reference biomarker score by subtracting the reference biomarker score from the cardiac biomarker score. When binary values for the risk predictors are used, the reference biomarker score will generally be zero. A positive difference between the cardiac biomarker score and the reference biomarker score indicates the subject has an increased risk of developing cardiovascular disease compared to the baseline risk of developing cardiovascular disease that is present in a reference population. The cardiac biomarker score essentially represents a control value for the risk indicators used for a particular population of subjects, such as the general population.

If the cardiac biomarker score is greater than the reference biomarker score, the test subject is at greater risk of developing or having CVD than individuals with levels comparable to or below the control value or in the lower range of control values. In contrast, if the cardiac biomarker score in the test subject's biological sample is below the reference biomarker score, the test subject is at a lower risk of developing or having CVD than individuals whose levels are comparable to or above the control value or exceeding or in the upper range of control values. The extent of the difference between the test subject's risk predictor levels and control value is also useful for characterizing the extent of the risk and thereby determining which individuals would most greatly benefit from certain aggressive therapies.

Control values are based upon the level of the risk predictor (e.g., BNP, MPO, and/or hsCRP) in comparable samples obtained from a reference cohort. In certain embodiments, the reference cohort is the general population. For example, the reference cohort can be a select population of human subjects. In certain embodiments, the reference cohort is comprised of individuals who have not previously had any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. In certain embodiments, the reference cohort is comprised of individuals, who if examined by a medical professional would be characterized as free of symptoms of disease.

In another example, the reference cohort may be individuals who are smokers, or alternately who are nonsmokers. A nonsmoker cohort may have a different normal range of the risk predictors being used than will a smoking population or the general population. A reference cohort can also be made up of individuals known to be diabetic or pre-diabetic. For example, if the subject is diabetic, a diabetic risk profile can be used, and if the subject is pre-diabetic, a pre-diabetic risk profile can be used. Accordingly, the control values selected may take into account the category into which the test subject falls. Appropriate categories can be selected with no more than routine experimentation by those of ordinary skill in the art.

The control value is provided in a manner that corresponds or relates to the value used to characterize the level of risk predictors obtained from the test subject. Thus, if the level of the BNP, MPO, or hsCRP is an absolute value such as the units of BNP, MPO, or hsCRP per ml of blood, the control value is also based upon the units of BNP, MPO, or hsCRP per ml of blood in individuals in the general population or a select population of human subjects.

The control value can take a variety of forms. The control value can be a single cut-off value, such as a median or mean. Control values of risk predictors in biological samples obtained, such as for example, mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each risk predictor that is assayed. A standardized method that may be used employs the guaiacol oxidation assay as described by Klebanoff et al., Methods in Enzymology. 105: 399-403 (1984).

In some embodiments, a predetermined value is used. A predetermined value can be based on the levels of risk predictor in a biological sample taken from a subject prior to cardiovascular therapeutic intervention, such as administration of a cardiovascular therapeutic agent. In another embodiment, the predetermined value is based on the levels of the risk predictor in biological samples taken from control subjects that are apparently healthy, as defined herein. A predetermined value can include levels present in subjects having been diagnosed as having cardiovascular disease. Unlike control values, predetermined values can be individualistic and need not be based on sampling of a population of subjects.

The cardiac biomarker score for a subject can be compared to a range of and the reference biomarker scores to provide risk stratification for a subject. Risk stratification, as used herein, refers to characterization of the risk into a number of different risk categories, such as low risk, moderate risk, and high risk. Risk stratification thus provides a finer grained answer regarding the level of risk than certain other embodiments of the invention. Risk stratification is obtained by identifying where the subject's cardiac biomarker score falls within a risk profile range. For example, a risk profile range can be provided that provides various ranges of reference biomarker scores and correlates them with differing levels of risk. The risk profile can be established based upon comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having CVD can be based upon which group his or her test value falls. For example, if binary risk predictor differentials are used with three different risk predictors, a risk profile range in which a cardiac biomarker score of 0 indicates normal risk, a cardiac biomarker score of 1 indicates low risk, a cardiac biomarker score of 2 indicates moderate risk, and a cardiac biomarker score of 3 indicates high risk.

The effectiveness of a diagnostic method can be evaluated using a net reclassification index. The next reclassification index represents how many subjects move from one risk classification level to another (i.e., from low to medium risk) and is a measure of how much of an impact the diagnostic method has on evaluation of risk for subjects. For example, use of a cardiac biomarker score according to the method described herein can provide a net risk classification index of 10% or more.

In certain embodiments, the subject's risk profile for CVD is determined by combining a first risk value, which is obtained by comparing levels of risk predictors in a biological sample of the subject with levels of corresponding risk predictors in a control population, with one or more additional risk values to provide a final cardiac biomarker score. Such additional risk values may be obtained by procedures including, but not limited to, determining the subject's blood pressure, assessing the subject's response to a stress test, low density lipoprotein, or cholesterol in a biological sample from the subject, or assessing the subject's atherosclerotic plaque burden.

### Therapeutic Methods

The present invention also relates to methods of identifying a subject in need of a cardiovascular therapeutic intervention to reduce the risk of developing cardiovascular disease or a complication thereof. The cardiovascular therapeutic intervention can be surgery, administration of a therapeutic agent, or implementation of a beneficial cardiovascular life style change by the subject. The method can include recommendation of a cardiovascular therapeutic intervention, or it can actually include the intervention itself. In some embodiments, levels of a plurality of biomarkers selected from BNP, MPO, and hsCRP are assessed at one or more time points following therapy to monitor the effectiveness of the therapy and, as desired, to alter the therapy accordingly (e.g., continue therapy, discontinue therapy, change therapy).

The present predictive tests are useful for determining if and when therapeutic agents that are targeted at preventing CVD or for slowing the progression of CVD should and should not be prescribed for a subject. For example, subjects with a cardiac biomarker score above a certain cutoff value, or that are in the higher tertile or quartile of a “normal range,” could be identified as those in need of more aggressive intervention with lipid lowering agents, life style changes, etc. It is particularly desirable to provide cardiac therapeutic intervention if a subject has been identified as being at high risk.

In one embodiment, the method comprises recommending administration or administering to the subject identified as having an elevated cardiac biomarker score a suitable cardiovascular agent. Examples of cardiovascular agents include an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, a calcium channel blocker, a beta-adrenergic receptor blocker, a cyclooxygenase-2 (COX-2) inhibitor, an angiotensin system inhibitor, or combinations thereof. The agent is administered in an amount effective to lower the risk of the subject developing a future cardiovascular disorder. A wide variety of cardiovascular agents together with their recommended dosages, pharmacology, and contraindications can be found in the most recent version of the Physician's Desk Reference (currently the 59th edition), which is incorporated herein by reference.

In a further embodiment, the method includes recommending and/or conducting a surgical intervention for the subject such as coronary angioplasty, coronary atherectomy, ablative laser-assisted angioplasty, catheter-based thrombolysis, mechanical thrombectomy, coronary stenting, coronary radiation implant, coronary brachytherapy (delivery of beta or gamma radiation into the coronary arteries), and coronary artery bypass surgery.

In another embodiment, the method includes recommendation of and/or implementation of a beneficial life style change by the subject. Lifestyle changes include, for example, weight loss, a diet modification such as practicing a low saturated fat, low cholesterol, and/or low sodium diet, regular exercise, and a prohibition on smoking.

### Evaluation of Therapeutic Intervention

Another embodiment of the invention provides a method for evaluating the efficacy of cardiovascular therapeutic intervention in a subject with cardiovascular disease. The therapeutic intervention can be any of the various types of therapeutic invention described herein, such as the use of cardiovascular agents, life style changes, and surgical intervention. The method includes determining levels of a plurality of risk predictors in a biological sample taken from the subject prior to therapy and determining the level of the corresponding risk predictors in a biological sample (e.g., an equivalent sample) taken from the subject during or following therapy. A decrease in overall levels of the risk predictors in the sample taken after or during therapy as compared to corresponding levels of risk predictors in the sample taken before therapy is indicative of a positive effect of the therapy on cardiovascular disease in the treated subject.

Also provided are methods for evaluating the effect of CVD therapeutic agents on individuals who have been diagnosed as having or as being at risk of developing CVD. Such therapeutic agents include, but are not limited to, anti-inflammatory agents, insulin sensitizing agents, antihypertensive agents, anti-thrombotic agents, anti-platelet agents, fibrinolytic agents, lipid reducing agents, direct thrombin inhibitors, ACAT inhibitor, CDTP inhibitor thioglitazone, glycoprotein II b/IIIa receptor inhibitors, agents directed at raising or altering HDL metabolism such as apoA-I milano or CETP inhibitors (e.g., torcetrapib), or agents designed to act as artificial HDL. Such evaluation comprises determining the levels of a plurality of biomarkers in a biological sample taken from the subject prior to administration of the therapeutic agent and a corresponding biological fluid taken from the subject following administration of the therapeutic agent. A decrease in the level of the selected risk markers in the sample taken after administration of the therapeutic as compared to the level of the selected risk markers in the sample taken before administration of the therapeutic agent is indicative of a positive effect of the therapeutic agent on cardiovascular disease in the treated subject.

### Kits

The present disclosure also provides kits for assaying samples for presence and amount of the risk factors (e.g., B-type natriuretic peptide (BNP), myeloperoxidase (MPO), and high-sensitivity C-reactive protein (hsCRP) and optionally one or more additional risk predictors of cardiovascular disease. Such kits may include one or more reagents useful for performing one or more immunoassays for detection and quantification of BNP, MPO, and hsCRP and any one or more additional biomarkers. Accordingly, the kit can include a plurality of reagents selected from the group consisting of: a reagent capable of detecting B-type natriuretic peptide (BNP), a reagent capable of detecting myeloperoxidase (MPO), and a reagent capable of detecting high-sensitivity C-reactive protein (hsCRP). In some embodiments, the kit includes a reagent capable of detecting B-type natriuretic peptide (BNP), a reagent capable of detecting myeloperoxidase (MPO), and a reagent capable of detecting high-sensitivity C-reactive protein (hsCRP). The kit also includes plurality of reference values or control samples suitable for use with the selected reagents. For example, the kit can further comprising at least one additional reagent capable of detecting low density lipoprotein, cholesterol, apolipoprotein A1, apolipoprotein B100, or creatinine.

A kit generally includes a package with one or more containers holding the reagents, as one or more separate compositions or, optionally, as an admixture where the compatibility of the reagents will allow. The kit can also include other material(s), which may be desirable from a user standpoint, such as a buffer(s), a diluent(s), a standard(s), and/or any other material useful in sample processing, washing, or conducting any other step of the assay.

In some embodiments, the reagents include antibodies capable of specifically binding to the compound they are capable of detecting. For example, the kit can include an antibody specific for BNP, an antibody specific for MPO, and/or an antibody specific for hsCRP. The kit can also include one or more antibodies each specific for any additional risk predictors being used. Antibody reagents can be used as a positive control in immunoassays detecting the risk predictors. If desired, multiple concentrations of each antibody can be included in the kit to facilitate the generation of a standard curve to which the signal detected in the test sample can be compared. Alternatively, a standard curve can be generated by preparing dilutions of a single antibody solution provided in the kit.

As used herein, the terms “specific binding” or “specifically binding”, refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “antibody” refers to an immunoglobulin molecule or immunologically active portion thereof, namely, an antigen-binding portion. Examples of immunologically active portions of immunoglobulin molecules include F(ab) and F(ab′) 2 fragments which can be generated by treating an antibody with an enzyme, such as pepsin. Examples of antibodies that can be used in the present disclosure include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above.

Test kits according to the present disclosure may also include a solid phase, to which the antibodies functioning as capture antibodies and/or detection antibodies in a sandwich immunoassay format are bound. The solid phase may be a material such as a magnetic particle, a bead, a test tube, a microtiter plate, a cuvette, a membrane, a scaffolding molecule, a quartz crystal, a film, a filter paper, a disc or a chip. The kit may also include a detectable label that can be or is conjugated to an antibody, such as an antibody functioning as a detection antibody. The detectable label can for example be a direct label, which may be an enzyme, oligonucleotide, nanoparticle chemiluminophore, fluorophore, fluorescence quencher, chemiluminescence quencher, or biotin. Test kits may optionally include any additional reagents needed for detecting the label.

The kit can also include instructions for using the kit to carry out a method of characterizing the risk for cardiovascular disease for a subject using the reagents and the reference values or control samples. In further embodiments, the method provides risk stratification for cardiovascular disease for a subject. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

An example has been included to more clearly describe a particular embodiment of the invention and its associated cost and operational advantages. However, there are a wide variety of other embodiments within the scope of the present invention, which should not be limited to the particular example provided herein.

### EXAMPLE

### Usefulness of Cardiac Biomarker Score for Risk Stratification in Stable Patients Undergoing Cardiac Evaluation across Glycemic Status

The inventor prospectively evaluated 3,635 consecutively consented subjects undergoing elective cardiac catheterization recruited between 2001 and 2006 without evidence of myocardial infarction (cardiac troponin I [cTnI]<0.03 ng/mL). All participants gave written informed consent and the Institutional Review Board of the Cleveland Clinic approved the study protocol. The Framingham Risk Score was calculated for each subject based on the ATP III guidelines. JAMA 285:2486-2497 (2001). An estimate of creatinine clearance (CrCl) was calculated using the Cockcroft-Gault equation. Coronary artery disease was defined as any clinical history of myocardial infarction, percutaneous coronary intervention, coronary artery bypass surgery, or angiographic evidence of coronary artery disease (≧50% stenosis) in one or more major coronary arteries. Glycemic status and clinical definition of diabetes mellitus, “pre-diabetes,” and non-diabetes are defined by the latest practice guidelines based on fasting glucose and glycated hemoglobin levels (fasting glucose<100 mg/dL and HbA1c<5.7% for normals; fasting glucose≧126 mg/dL or HbA1c≧6.5% or currently taking glucose-lowering medications for diabetes mellitus; neither normal nor diabetes mellitus for pre-diabetes). Diabetes Care 35 Suppl 1:S11-63 (2012). Adjudicated outcomes were ascertained over the ensuing 3 years for all subjects following enrollment. The prospective determination of clinical outcomes is made by the research personnel contacting participants independent of study investigators, with pre-specified criteria and confirmation by review of documentation independent of the authors. Major adverse cardiovascular event (MACE) was defined as death, non-fatal myocardial infarction, or non-fatal cerebrovascular accident following enrollment. Blood samples were collected before administration of heparin, placed on ice, and processing, aliquotted and frozen at −80° F. within 2 hours of collection. All laboratory assays including hsCRP, BNP, MPO, apolipoprotein A1, apolipoprotein B100, and creatinine were performed using the Abbott ARCHITECT™ ci8200 platform (Abbott Diagnostics Inc, Abbott Park Ill.). The intra- and interassay coefficients were 4% and 2.4% for hsCRP, 2.6% and 3.5% for BNP, and 6.2% and 4.1% for MPO, respectively.

The Student's t-test or Wilcoxon-Rank sum test for continuous variables and chi-square test for categorical variables were used to examine the difference between the groups. A cardiac biomarker score was given to each group based on if it had a positive value in each respective 5 biomarker. Cutoffs were used for each of the three biomarkers (BNP>100 pg/mL, hsCRP>2.0 ng/L, and MPO>322 pmol/L) based upon prior cutoffs used for the respective markers as reported in prior studies. De Lemos et al., N Engl J Med; 345:1014-1021 (2001), Ridker et al., N Engl J Med; 342:836-843 (2000), Tang et al., Clinical chemistry; 57:33-39 (2011). Each of the groups was split into either 0, 1, 2 or 3 as a measure of how many of the biomarkers were deemed positive, which is defined as “cardiac biomarker score” (CBS). Kaplan-Meier analysis with Cox proportional hazards regression was used for time-to-event analysis to determine Hazard ratio (HR) and 95% confidence intervals (95% CI) for MACE. Unadjusted trends for all-cause mortality rates as well as non-fatal myocardial infarction/stroke rates with increasing quartiles of MPO, hsCRP, and BNP were evaluated with the Cochran-Armitage test using a time-to-event approach. Adjustments were made for individual traditional cardiac risk factors (including age, gender, low-density and high-density lipoprotein cholesterol, systolic blood pressure, former or current cigarette smoking, diabetes mellitus, apolipoprotein B100/apolipoprotein A1 ratio, history of myocardial infarction, and creatinine clearance [CrCl]) to predict incident 3-year MACE risks. Net reclassification analysis was performed with both Cox models adjusted for traditional risk factors. Cutoff values for net reclassification index estimation used a ratio of 6:3:1 for low, medium and high risk categories. All analyses were performed using R (Vienna 8.02). P values<0.05 were considered statistically significant.

Table 1 describes the baseline characteristics of the study population, and is stratified according to glycemic status. The median levels of hsCRP, BNP, and MPO were 2.00 [interquartile range 0.91-4.47] pg/mL, 83 [interquartile range 34-200] pg/mL, and 103 [interquartile range 70-195] pmol/L, respectively. All 3 biomarkers were notably elevated in diabetic patients as compared to those with pre-diabetes or non-diabetes. Table 2 represents the prognostic value of individual cardiac biomarkers in the study cohort. All 3 cardiac biomarkers provided incremental risk prediction in the study cohort (Table 2). After adjusting for traditional risk factors including Framingham risk factors, log transformed BNP, hsCRP, and MPO each remained independent predictors of incident major adverse cardiac events (MACE) at 3-year follow-up (Table 2).

TABLE 1
Baseline Characteristics

| | Whole | Diabetes | Pre- | Non- | |
|---|---|---|---|---|---|
| | cohort | mellitus | diabetes | diabetes | |
| Variable | (n = 3635) | (n = 1014) | (n = 1529) | (n = 1092) | p-value |
| Age (years) | 63 ± 11 | 64 ± 10 | 63 ± 11 | 61 ± 12 | <0.001 |
| Male | 65% | 61% | 70% | 64% | <0.001 |
| Hypertension | 71% | 78% | 70% | 62% | <0.001 |
| History of MI | 33% | 35% | 32% | 32% | <0.150 |
| Median systolic blood | 133 | 134 | 132 | 132 | <0.015 |
| pressure (mmHg) | (120, 146) | (120, 149) | (120, 145) | (119, 147) | |
| Low-density | 95 | 95 | 97 | 94 | <0.012 |
| lipoprotein cholesterol | (78, 116) | (77, 115) | (80, 118) | (76, 116) | |
| (mg/dL) | | | | | |
| High-density | 34 | 32 | 34 | 34 | <0.001 |
| lipoprotein cholesterol | (28, 41) | (27, 39) | (28, 42) | (29, 42) | |
| (mg/dL) | | | | | |
| Creatinine clearance | 100 | 99 | 100 | 100 | <0.512 |
| (ml/min/1.73 m²) | (76, 126) | (74, 128) | (77, 126) | (79, 126) | |
| Cigarette smoking | 65% | 64% | 68% | 62% | <0.002 |
| Aspirin | 73% | 73% | 74% | 71% | <0.357 |
| Beta-blockers | 61% | 65% | 62% | 56% | <0.001 |
| ACE inhibitors or | 50% | 60% | 47% | 41% | <0.001 |
| ARBs | | | | | |
| Statin | 59% | 63% | 59% | 54% | <0.001 |
| hsCRP (mg/L) | 2.00 | 2.56 | 1.89 | 1.67 | <0.001 |
| | (0.91, 4.47) | (1.13, 5.93) | (0.86, 3.95) | (0.83, 4.00) | |
| BNP (pg/mL) | 83 | 93 | 78 | 83 | <0.001 |
| | (34, 200) | (40, 240) | (32, 177) | (32, 198) | |
| MPO (pmol/L) | 103 | 105 | 104 | 100 | <0.199 |
| | (70, 195) | (74, 186) | (69, 201) | (68, 194) | |

Abbreviations:
MI = myocardial infarction,
ACE = angiotensin converting enzyme,
ARB = angiotensin receptor blocker,
hsCRP = high sensitivity C-reactive protein,
BNP = B-type natriuretic peptide,
MPO = myeloperoxidase.

TABLE 2
Cox proportional hazards analyses for individual cardiac biomarker
and future major adverse cardiac events at 3 years

| | Univariate Model | | Multivariate Model * | |
|---|---|---|---|---|
| | Hazard Ratio | | Hazard Ratio | |
| | (95% | | (95% | |
| | Confidence | | Confidence | |
| Variable | Interval) | P-value | Interval) | P-value |
| BNP > 100 pg/mL | 2.76 (2.25-3.39) | <0.001 | 2.10 (1.65-2.68) | <0.001 |
| hsCRP > 2 ng/L | 2.10 (1.71-2.58) | <0.001 | 1.82 (1.46-2.28) | <0.001 |
| MPO > 322 pmol/L | 1.43 (1.12-1.82) | <0.004 | 1.32 (1.02-1.71) | <0.036 |

* Adjusted for age, gender, low-density and high-density lipoprotein cholesterol, systolic blood pressure, former or current cigarette smoking, diabetes mellitus, and history of myocardial infarction, creatinine clearance. Abbreviations are as in Table 1.

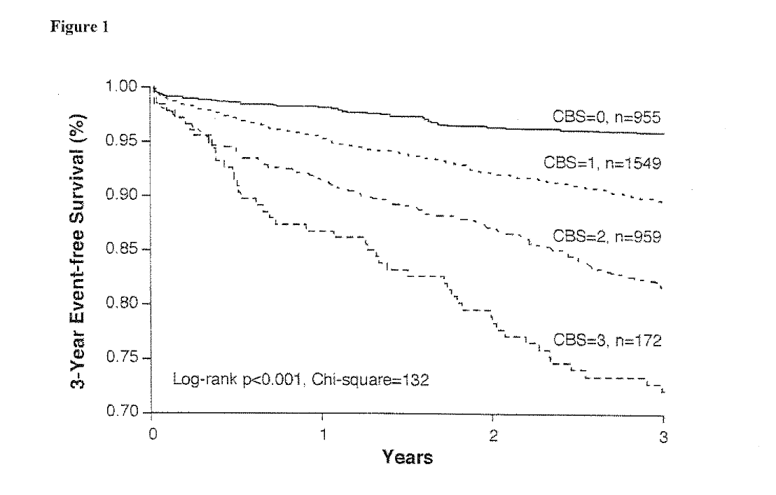

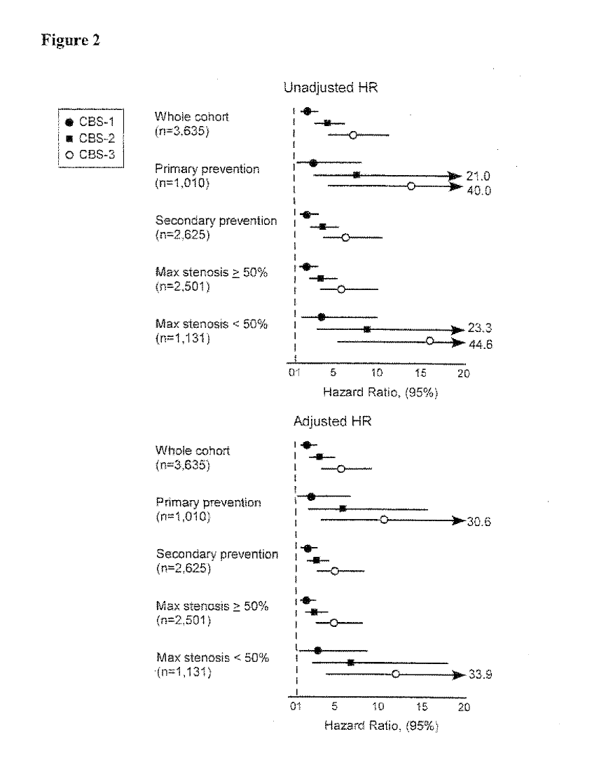

By summing up the number of positive cardiac biomarkers, a cardiac biomarker score (CBS) was developed that integrates the risk profile of the study cohort. As illustrated in FIG. 1, CBS provides incremental prognostic value as displayed by Kaplan-Meier analysis. In Table 3, a CBS based on sum total of “positive” biomarkers provided independent prediction of future risk of incident MACE at 3 years (HR: 7.61 [95% CI: 4.98-11.65] p<0.001), even after adjusted for traditional risk factors (6.11 [95% CI: 3.98-9.38] p<0.001), in addition to ApoB/ApoA1 ratio (6.11 [95% CI: 3.98-9.38] p<0.001)(Table 3). The ability for CBS to provide incremental risk stratification can be seen in subgroups of patients with primary and secondary prevention, as well as those with maximal stenosis of <50% and ≧50% of their coronary arteries (FIG. 2). Higher CBS predicted future risk of MACE at 3 years regardless of age, gender, body mass index, diabetes mellitus, hypertension, renal insufficiency, or prior myocardial infarction (all p<0.01). Use of CBS on top of traditional risk factors was also shown to reclassify subjects (Net reclassification index 12.86%, p<0.001; Integrated Discrimination Improvement 12.0%, p<0.001; C-statistics 66.9% vs. 71.1%, p<0.001).

TABLE 3
Cox proportional hazard analyses of cardiac biomarker score stratified by glycemic status

| | Cardiac Biomarker Score | | | |
|---|---|---|---|---|
| | 0 | 1 | 2 | 3 |
| Whole cohort (n = 3,635) | | | | |
| Unadjusted HR | 1 | 2.59 (1.82-3.68)** | 4.72 (3.33-6.69)** | 7.61 (4.98-11.65)** |
| Adjusted HR(1) | 1 | 2.27 (1.59-3.23)** | 3.67 (2.58-5.24)** | 6.11 (3.98-9.38)** |
| Adjusted HR(2) | 1 | 2.27 (1.59-3.23)** | 3.67 (2.58-5.24)** | 6.11 (3.98-9.38)** |
| MACE events | 39/955 | 159/1549 | 173/959 | 47/172 |
| Normal (n = 1,014) | | | | |
| Unadjusted HR | 1 | 1.78 (0.94-3.37) | 3.23 (1.69-6.16)** | 6.23 (2.9-13.37)** |
| Adjusted HR(1) | 1 | 1.43 (0.74-2.75) | 2.37 (1.21-4.64)* | 4.73 (2.18-10.25)** |
| Adjusted HR(2) | 1 | 1.36 (0.7-2.61) | 2.23 (1.14-4.39)* | 4.24 (1.96-9.18)** |
| MACE events | 13/290 | 35/445 | 32/230 | 13/49 |
| Pre-diabetic (n = 1,529) | | | | |
| Unadjusted HR | 1 | 2.74 (1.56-4.84)** | 4.75 (2.7-8.38)** | 10.27 (5.24-20.13)** |
| Adjusted HR(1) | 1 | 2.4 (1.36-4.23)** | 3.67 (2.06-6.54)** | 7.87 (3.99-15.55)** |
| Adjusted HR(2) | 1 | 2.37 (1.35-4.19)** | 3.58 (2-6.41)** | 7.62 (3.87-15.01)** |
| MACE events | 15/427 | 61/654 | 61/379 | 20/69 |
| Diabetes Mellitus (n = 1,092) | | | | |
| Unadjusted HR | 1 | 3.17 (1.67-6)** | 5.59 (2.98-10.49)** | 6.16 (2.8-13.52)** |
| Adjusted HR(1) | 1 | 3.06 (1.61-5.78)** | 4.79 (2.55-9)** | 5.59 (2.54-12.31)** |
| Adjusted HR(2) | 1 | 3.05 (1.61-5.77)** | 4.8 (2.55-9.01)** | 5.61 (2.55-12.33)** |
| MACE events | 11/238 | 63/450 | 80/350 | 14/54 |

Model 1: adjusted for traditional risk factors include age, gender, systolic blood pressure, low-density lipoprotein cholesterol, high-density lipoprotein cholesterol, smoking
Model 2: Adjusted for traditional risk factors plus apolipoprotein B100/apolipoprotein A1 ratio
**p < 0.001
Abbreviations:
HR = hazard ratio,
MACE = major adverse cardiovascular events.

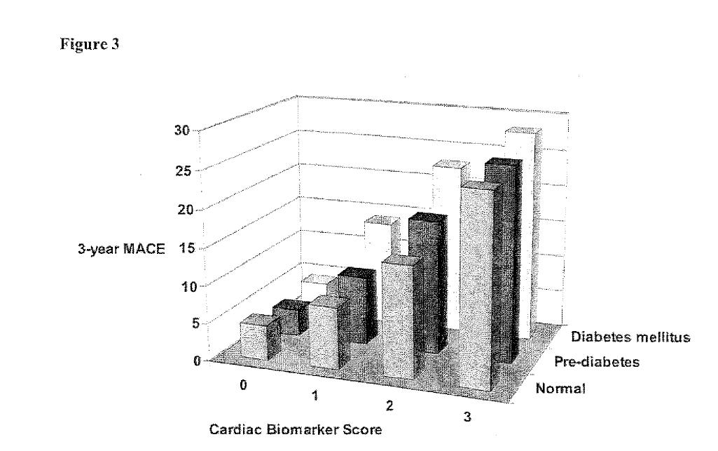

The study cohort was further analyzed according to glycemic status and different subgroups. The capability of CBS to stratify patients' risk profiles within subjects with diabetes mellitus, pre-diabetes, or normoglycemic (non-prediabetic and non-diabetic) based on practice guidelines remains robust (Table 3, FIG. 3). In a similar manner, after adjustment with HbA1c, the prognostic value of CBS was preserved. Furthermore, the prognostic value of CBS was similar regardless of age, gender, body mass index, diabetes mellitus, hypertension, renal insufficiency, or prior myocardial infarction (all p<0.01).

### Discussion

While previous studies have examined similar multimarker strategies for risk prediction, many of them utilized biomarkers that are not commonly used or available in the clinical practice settings. The key finding in this study is the incremental prognostic value of all three clinically available plasma cardiac biomarkers beyond standard evaluation of classic Framingham risk factors, renal function, and apolipoprotein B100/apolipoprotein A1 ratio in a troponin-negative, stable cardiac patients undergoing coronary angiography. Comparable prognostic value was further identified within subsets of patients with pre-diabetes or non-diabetes, or those with no significantly obstructive coronary artery disease, further underscores the potential for a multimarker approach in identifying vulnerable patients within cohorts that may allow for targeting risk factor modifications and more aggressive preventive interventions.

High sensitivity C-reactive protein is the most common systemic inflammatory biomarker utilized in clinical practice, particularly in patients with diabetes mellitus and potential response to statin therapy. Ridker et al., N Engl J Med; 344:1959-1965 (2001). Ridker et al., N Engl J Med; 347:1557-1565 (2002). Elevated levels have also been associated with altered cardiac structure and function, as well as adverse long-term consequences. Tang et al., Am J Cardiol; 101:370-373 (2008). In addition, hsCRP has been suggested to play a role in atherosclerosis and its complications, though genetic studies suggest the association with adverse outcomes may not be causal. Anand et al., Eur Heart J; 31:2092-2096 (2010). Elliott et al., JAMA; 302:37-48 (2009).

In contrast, MPO has been shown to directly promote catalytic consumption of nitric oxide, leading to the development of endothelial dysfunction. Vita et al., Circulation; 110:1134-1139 (2004). MPO is a leukocyte-derived haemoprotein that has been linked in the development and subsequent instability of atherosclerotic plaques. Nicholls et al., Arterioscler Thromb Vasc Biol; 25:1102-1111 (2005). Previous studies have shown MPO to have prognostic significance among subjects with unstable angina, as well as following acute myocardial infarction, acute heart failure, and chronic stable heart failure as well as healthy middle aged or elderly subjects. Tang et al., Congest Heart Fail; 17:105-109 (2011). Nicholls et al., Clin Chem; 57:1762-1770 (2011). Reichlin et al., Clin Chem; 56:944-951 (2010). Naruko, Heart; 96:1716-1722 (2010). Nicholls S J, Hazen S L. Arteriosclerosis, Thrombosis, and Vascular Biology; 25:1102-1111 (2005). Brennan et al., The New England Journal of Medicine; 349:1595-1604 (2003). Recently, it has also been found that MPO remained a statistically significant prognostic indicator of cardiovascular risk in a large stable CAD population. Tang et al., Clinical chemistry; 57:33-39 (2011).

On the other hand, the natriuretic peptide family is a group of endogenous peptides primarily produced in the heart that provide counter-regulatory effects on a wide range of organs to maintain perfusion and reduce overloading status of the vasculature. Tang W H, Congest Heart Fail; 13:48-52 (2007). BNP has recently been shown to be elevated in acute coronary syndromes without necessarily having myocardial infarction, and may reflect not only the underlying impairment of left ventricular function but also the severity of the ischemic episode. Fonarow et al., Journal of the American College of Cardiology; 49: 1943-1950 (2007). Altogether, this combination of biomarkers offer complimentary mechanistic insights during cardiac evaluation in stable patients, even though only a small subset of patients demonstrated “positive” for all 3 biomarkers in the relatively stable patient cohort.

The study further explores the impact of glycemic control on prognostic value of cardiac biomarkers, particularly as the latest guidelines have highlighted a subset of “at-risk” patients that is thought to have heightened risk of developing diabetes mellitus and future cardiovascular risks. A graded increase in levels of each of the corresponding biomarkers was observed as patients were determined to be non-diabetic, pre-diabetic, and diabetic. The overall trend towards increased risk of MACE events was similar among all groups based on their CBS scores.

Similarly, CBS provided significant prognostic value amongst subjects for whom no significant angiographic evidence of stenosis was discovered, and most often considered having lower risk. A strength of this study is the considerable size of the patient population. This contemporary cohort of stable cardiac patients is representative current clinical practice. The focus on the homogeneous elective coronary angiography population and the availability and inclusion of only FDA-cleared biomarker data for the analyses strengthen the study, as the present biomarkers, while clinically available for use, are not routinely measured. Including them in the analysis provides insight into a non-acute, troponin-negative population, which has yet to be thoroughly investigated. Incremental contributions of these cardiac biomarkers towards risk stratification above and beyond standard clinical and biochemical characteristics in this population have also not been thoroughly tested, particularly with rigorous statistical evaluation or covariate adjustments

Potential weaknesses of the study population arise because a clinical trial cohort undergoing coronary angiography was used and thus there may be referral bias particularly as they are already undergoing cardiac evaluation. Moreover the data relate to prognostic rather than diagnostic applications of these biomarkers. High sensitivity troponin assays were not used in the cohort that was deemed “troponin-negative” by currently approved troponin assays. Lastly, while the results were based on previously used cut points, they may overestimate the strengths of the risk relationships. With different studies using different cutoff values for different populations, there is a need to identify clinically-useful cut points based on consensus of results.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of characterizing the risk for developing cardiovascular disease comprising:
determining the levels of a plurality of risk predictors in a biological sample obtained from a subject using an analytic device, wherein the risk predictors are selected from the group consisting of B-type natriuretic peptide (BNP), myeloperoxidase (MPO), and high-sensitivity C-reactive protein (hsCRP);
comparing the levels of the plurality of risk predictors to corresponding control values to obtain a risk predictor differential for each risk predictor;
adding the plurality of risk predictor differentials to provide a cardiac biomarker score; and comparing the cardiac biomarker score to a reference biomarker score, wherein a positive difference between the cardiac biomarker score and the reference biomarker score indicates the subject has an increased risk of developing cardiovascular disease compared to the risk of a reference population.

2. The method of claim 1, wherein the amount of the positive difference between the cardiac biomarker score and the reference biomarker score correlates with the level of increased risk of developing cardiovascular disease.

3. The method of claim 1, wherein the method of characterizing the risk for developing cardiovascular disease comprises risk stratification, and the risk stratification is obtained by identifying where the subject's cardiac biomarker score falls within a risk profile range.

4. The method of claim 1, wherein each risk predictor that exceeds the control value is designated a positive risk predictor having a risk predictor differential of 1 and all other risk predictors are designated as null risk predictors having a risk predictor differential of 0.

5. The method of claim 1, wherein the risk predictors comprise BNP and MPO.

6. The method of claim 1, wherein the risk predictors comprise BNP and hsCRP.

7. The method of claim 1, wherein the risk predictors comprise MPO and hsCRP.

8. The method of claim 1, wherein the risk predictors comprise BNP, MPO, and hsCRP.

9. The method of claim 3, wherein the subject is diabetic and a diabetic risk profile is used.

10. The method of claim 3, wherein the subject is pre-diabetic and a pre-diabetic risk profile is used.

11. The method of claim 1, wherein the step of determining the level of BNP further comprises determining the level of NT-pro-BNP.

12. The method of claim 1, wherein the biological sample is selected from the group consisting of blood, serum, plasma, and urine.

13. The method of claim 1, wherein the cardiovascular disease comprises a major adverse cardiac event.

14. The method of claim 1, wherein the analytic device is an ultraviolet spectrometer or mass spectrometer.

15. The method of claim 1, wherein the biological sample is stored before determining the levels of the plurality of risk predictors.

16. The method of claim 1, wherein one of the risk profile ranges is a high risk profile, and the method further comprises providing cardiovascular therapeutic invention to a subject identified as having a high risk profile.

17. The method of claim 16, wherein the cardiovascular therapeutic intervention is administration of a therapeutic agent.

18. The method of claim 16, wherein the cardiovascular therapeutic intervention is a beneficial cardiovascular life style change.

19. The method of method of claim 1, wherein the method further comprises one or more additional steps selected from
a) determining the subject's blood pressure;
b) determining the levels of low density lipoprotein, cholesterol, apolipoprotein A1, apolipoprotein B100, or creatinine in a biological sample from the subject;
c) assessing the subject's response to a stress test; and
d) determining the subject's atherosclerotic plaque burden;
wherein the results of the additional steps are factored into calculation of the cardiac biomarker score.

20. A kit comprising:
a plurality of reagents selected from the group consisting of:
a reagent capable of detecting B-type natriuretic peptide (BNP),
a reagent capable of detecting myeloperoxidase (MPO), and
a reagent capable of detecting high-sensitivity C-reactive protein (hsCRP);
a plurality of reference values or control samples suitable for use with the selected reagents, and a package holding the reagents.

21. The kit of claim 20, wherein the kit further comprises instructions for using the kit to carry out a method of characterizing the risk for cardiovascular disease for a subject using the reagents and the reference values or control samples.

22. The kit of claim 20, wherein the reagents are antibodies capable of specifically binding to the compound they are capable of detecting.

23. The kit of claim 20, wherein the reagents comprise a reagent capable of detecting B-type natriuretic peptide (BNP), a reagent capable of detecting myeloperoxidase (MPO), and a reagent capable of detecting high-sensitivity C-reactive protein (hsCRP).

24. The kit of claim 20, further comprising at least one additional reagent capable of detecting low density lipoprotein, cholesterol, apolipoprotein A1, apolipoprotein B100, or creatinine.
